(19) United States
(12) Patent Application Publication
(10) Pub. No.: US 2016/0213931 A1
(43) Pub. Date: Jul. 28, 2016

(54) METHOD OF SIGNAL GENERATION FOR NEUROMODULATION

(71) Applicant: Adam Sachs, Ottawa (CA)

(72) Inventor: Adam Sachs, Ottawa (CA)

(21) Appl. No.: 14/603,807

(22) Filed: Jan. 23, 2015

(51) Int. Cl. A61N 1/36

(52) U.S. Cl. CPC A61N 1/36175; A61N 1/36196

(57) ABSTRACT

A method of producing signals for neuromodulation has the steps of producing a starting signal using an oscillator, and shaping the frequency spectrum of the signal, wherein the signal is configured to be used for neuromodulation treatment. The starting signal is selected from the group consisting of a pulse wave, a sine wave, a triangle wave, a sawtooth wave and a reverse sawtooth wave. The signals may be added allowing the frequency spectrum of the signal to be shaped according to the formula:

$$f ( t ) = {a}_{0} + {\sum }_{k = 1}^{n} {a}_{k} {Ψ}_{k} ( {b}_{k} π t ) ,$$

The frequency spectrum of the signal may be shaped by pulse width modulation, frequency modulation, and one or more filters may be applied to the signal.

### FIELD OF THE INVENTION

The present invention relates generally to the field of electromagnetic signals used in neuromodulation and, more specifically, to shaping and controlling the generation of electromagnetic signals of the frequency spectrum of stimulation applied to the nervous system.

### BACKGROUND OF THE INVENTION

Typical neuromodulation signal generators output a square wave pulse. Examples include the internal pulse generators (IPG's) used in deep brain stimulators (DBS) and spinal cord stimulation (SCS). The electrical signals then interact with the patient's nervous system to produce the desired clinical effect. For example, patients with neuropathic limb pain benefit from chronic stimulation of the spinal cord in which appropriately chosen stimulation parameters selectively activate pathways that block the natural pain pathways, resulting in pain resolution for the patient. Similarly, patients with Parkinson's disease may benefit from electrodes implanted in the brain, which, during stimulation, restore the natural dynamics of motor circuitry that are impaired by the disease, resulting in improvement in movement. Currently available systems utilize a pulse wave in which the user, or health practitioner may modulate the frequency, amplitude, or duty cycle. The duty cycle represents the width of each pulse. When a square wave has a pulse width of 0.5, the signal is high half the time and at base level half the time.

The music industry has encountered an analogous need to generate sounds with rich, complex frequency and temporal attributes. Analog synthesizers have been used in music since the 1960s and permit the modulation of a musical output signal to perform operations such as pulse-width modulation, frequency and amplitude modulation, addition and subtraction of signals, filtering, filter cut-off modulation filter sweeps, and envelopes, to shape the temporal window of the waveform. A relatively simple envelope modifies the attack, delay, sustain and release of sounds. However, these synthesizer techniques have not been applied to neuromodulation for controlled generation and shaping of electromagnetic signals.

Therefore, there is a need in the art for a method of shaping neuromodulation signals in a manner analogous to a synthesizer's effect on sound, including pulse-width, frequency, and amplitude modulation; choice of waveform; addition of multiple waveforms; filtering including filters sweeps and cut-off; and affecting the attack, delay, sustain and release of sounds. Control over these stimulus attributes would result in substantially increased control over the frequency content delivered by neurostimulation devices, plausibly resulting in a higher degree of control over the physiological effect of neurostimulation, and consequently improved patient outcomes.

### SUMMARY OF THE INVENTION

A method of producing signals for neuromodulation has the steps of producing a starting signal using an oscillator, and shaping the frequency spectrum of the signal, wherein the signal is configured to be used for neuromodulation treatment. The starting signal may be selected from the group consisting of a pulse wave, a sine wave, a triangle wave, a sawtooth wave and a reverse sawtooth wave.

The frequency spectrum of the signal may be shaped according to the formula:

$$f ( t ) = {a}_{0} + {\sum }_{k = 1}^{n} {a}_{k} {Ψ}_{k} ( {b}_{k} π t ) ,$$

The frequency spectrum of the signal is shaped by pulse width modulation, or by frequency modulation. One or more filters may be applied to the signal, for example a lowpass, bandpass or highpass filter, and in an embodiment the filters are Butterworth filters, Chebyshev Type I filters, Chebyshev Type II filters and elliptic filters. A further step of applying pulse-width modulation to the signal may be added.

The foregoing, and other features and advantages of the invention, will be apparent from the following, more particular description of the preferred embodiments of the invention, the accompanying drawings, and the claims.

### BRIEF DESCRIPTION OF THE FIGURES

FIG. 1 shows a signal having variable pulse-width, according to an embodiment of the present invention;

FIG. 2 shows the power spectral density of a sine wave with a frequency of 100 Hz, according to an embodiment of the present invention;

FIG. 3A shows time-varying electrical signal of a pulse wave that has a frequency of 10 Hz, a duty cycle of 0.1 and a voltage of 1 V, in the prior art;

FIG. 3B shows the time-varying electrical signal of a pulse wave that has a square wave frequency of 10 Hz, a duty cycle of 0.2 and a fundamental voltage of 1 V, in the prior art;

FIG. 3C shows the time-varying electrical signal of a pulse wave that has a square wave frequency of 10 Hz, a duty cycle of 0.5 and a fundamental voltage of 1 V, in the prior art;

FIG. 4A shows the power spectral density of the pulse wave with a frequency of 10 Hz, a duty cycle of 0.1, in the prior art;

FIG. 4B shows the power spectral density of the pulse wave with a frequency of 10 Hz, a duty cycle of 0.2, in the prior art;

FIG. 4C shows the power spectral density of the pulse wave with a frequency of 10 Hz, a duty cycle of 0.5, in the prior art;

FIG. 5 shows the power spectral density of the pulse wave with a frequency of 10 Hz, a duty cycle of 0.3, a pulse width modulation of 20 Hz, and a pulse width modulation scale of 0.2, according to an embodiment of the present invention;

FIG. 6 replots the power spectral density in FIG. 5, on a scale from 0 to 100 Hz, as shown by line A in FIG. 6, according to an embodiment of the present invention;

FIG. 7 shows Matlab™ code for fundamental waveforms, according to an embodiment of the present invention; and

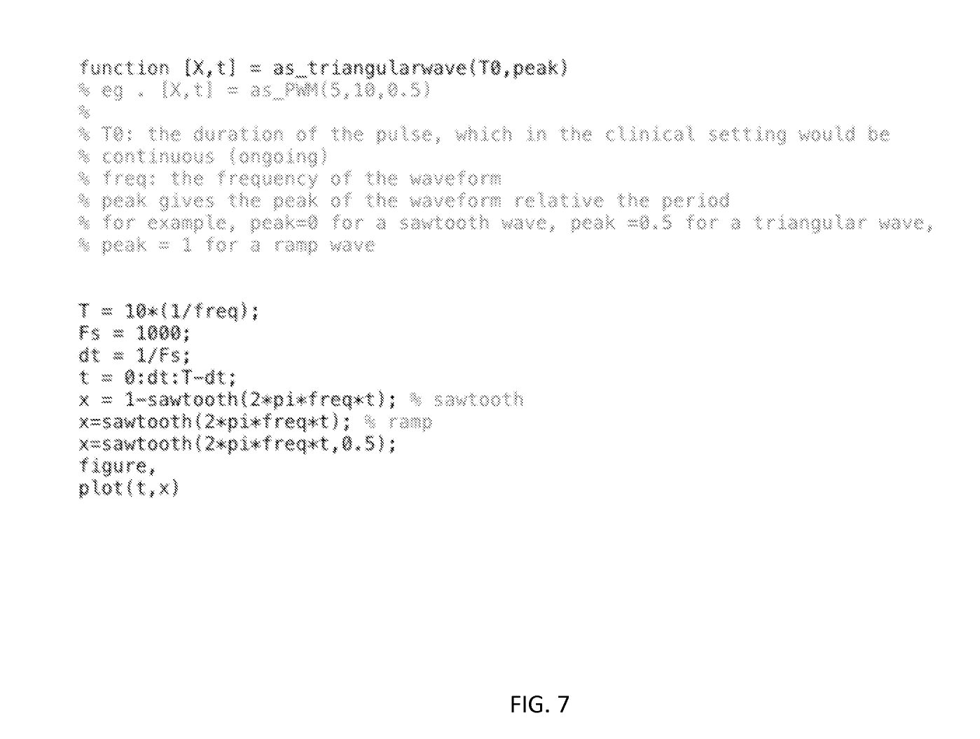

FIG. 8 shows Matlab™ code for pulse width modulation, according to an embodiment of the present invention.

### DETAILED DESCRIPTION

A method of generating and modifying electrical currents for neuromodulation is described. These currents will be produced by a programmable, battery powered, current generator, subject to digital or analogue processing steps as described in this patent. These “shaped” signals have different effects on the nervous system than prior art pulse (rectangular) waves. The nervous system has irregular firing behaviors with neural networks having highly irregular spectral densities with modulating frequency content. Therefore, clinical systems designed to intervene by inhibiting or exciting populations of neurons should be capable of producing current that can reflect this frequency-domain complexity.

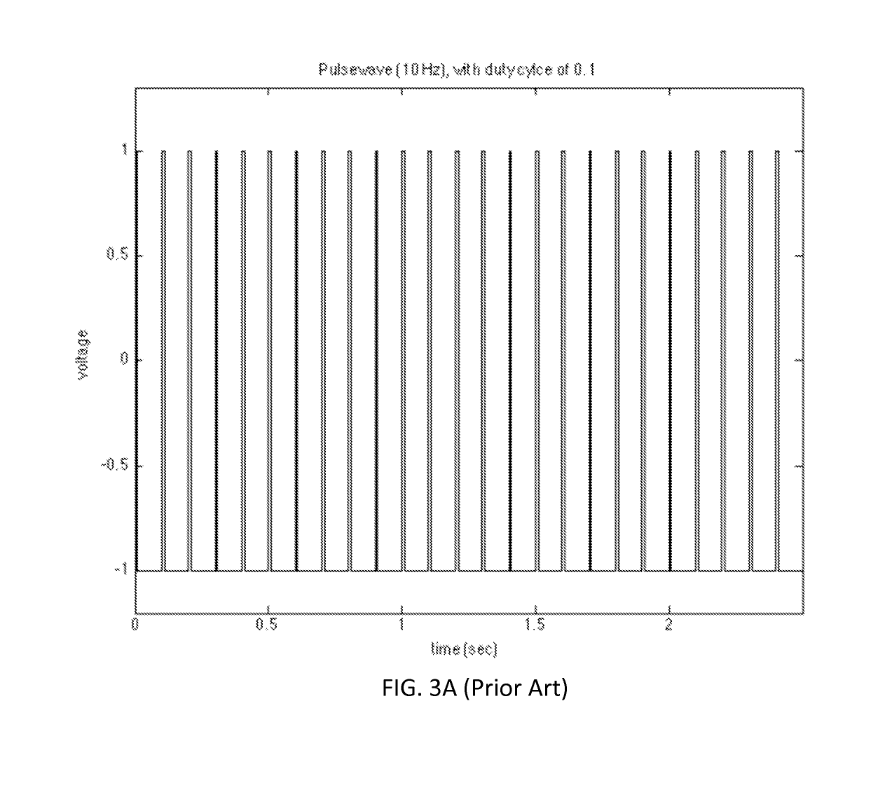

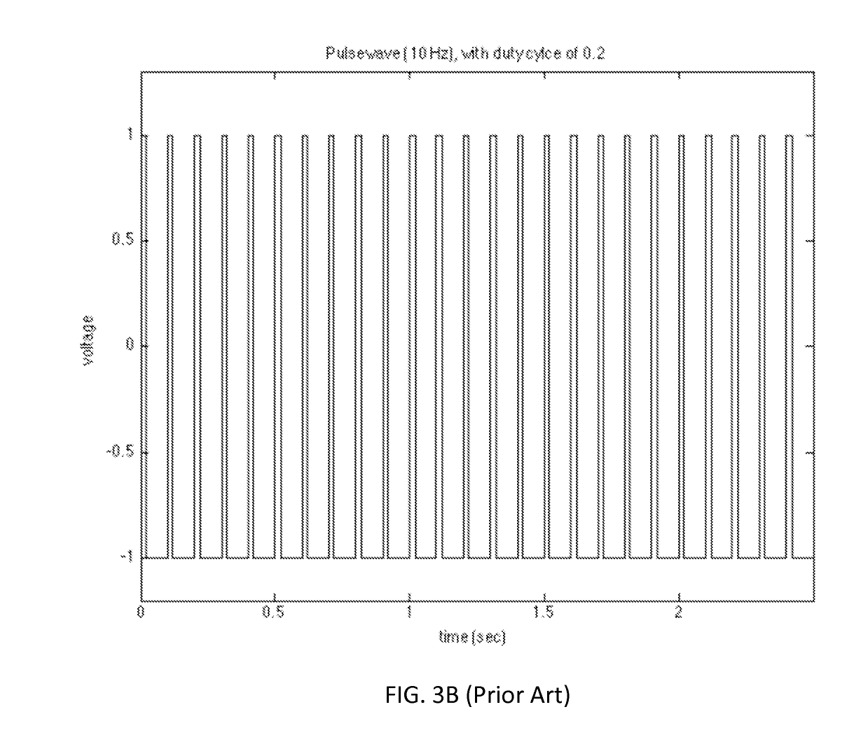

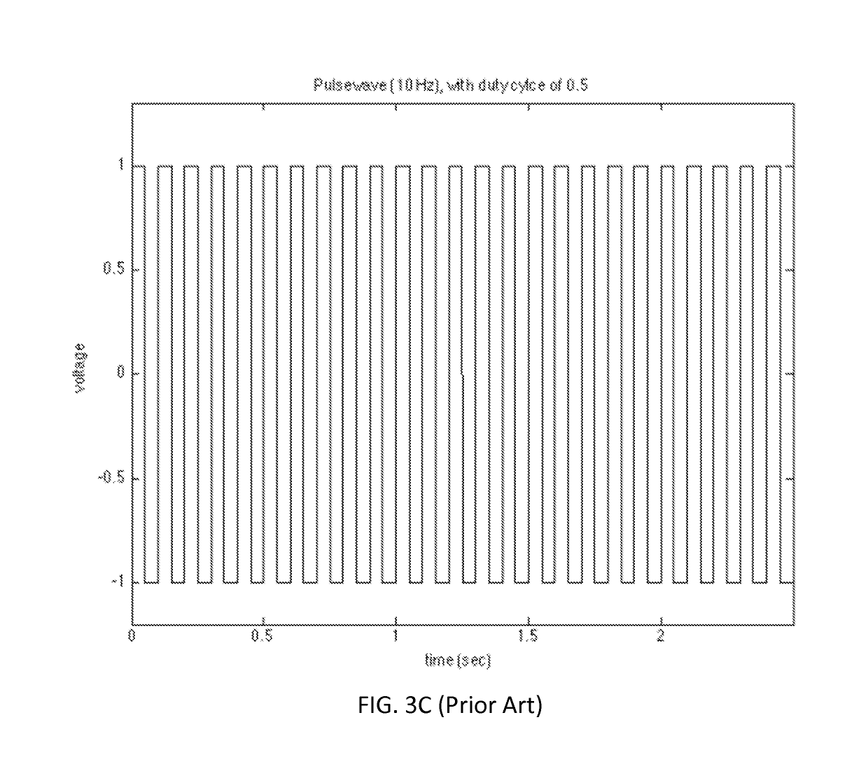

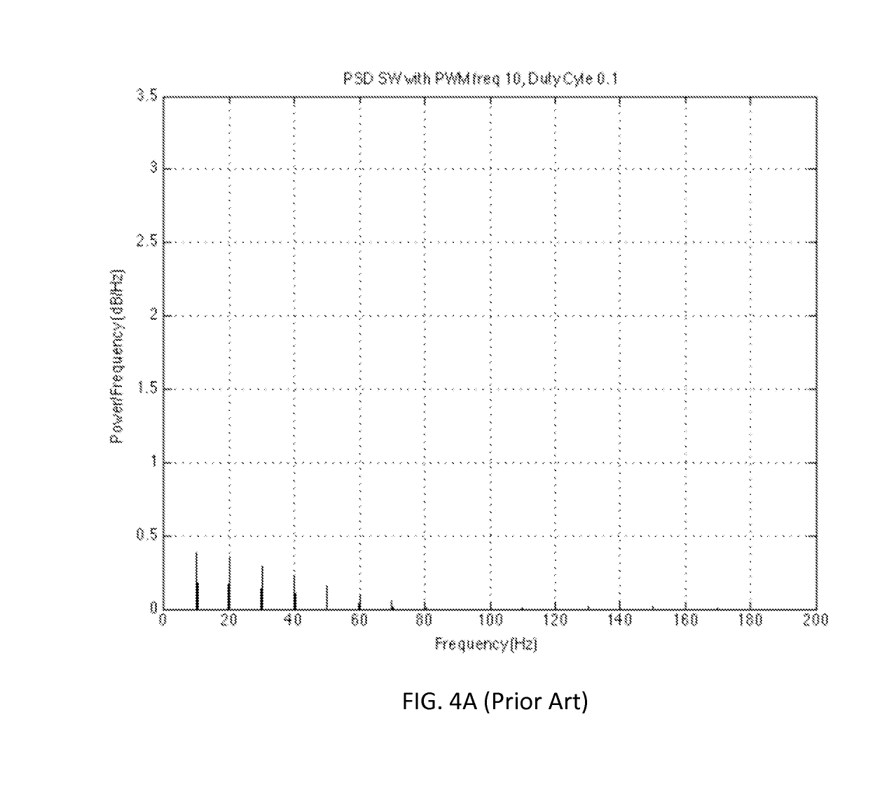

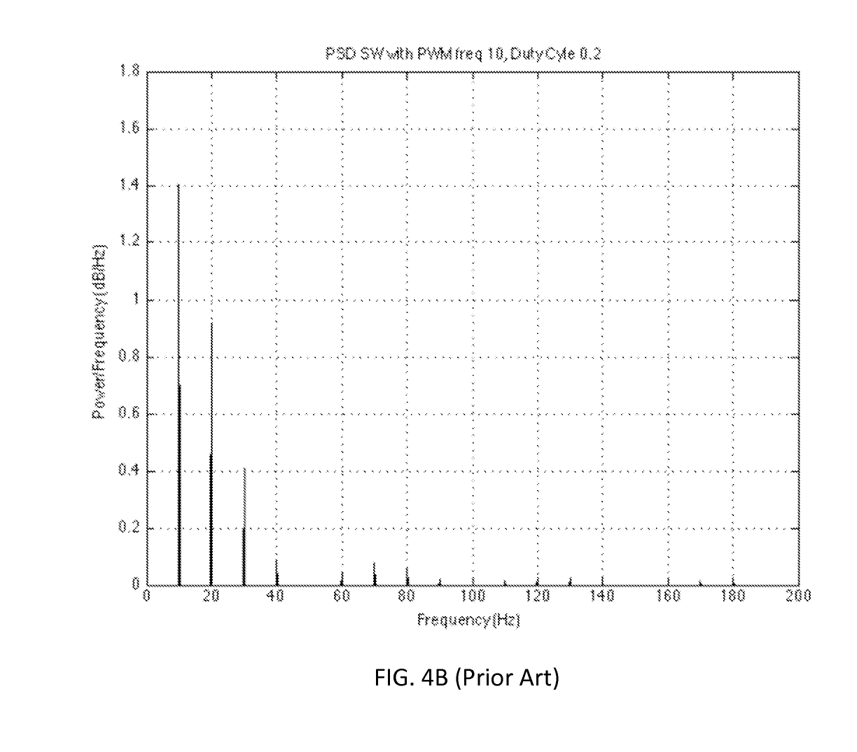

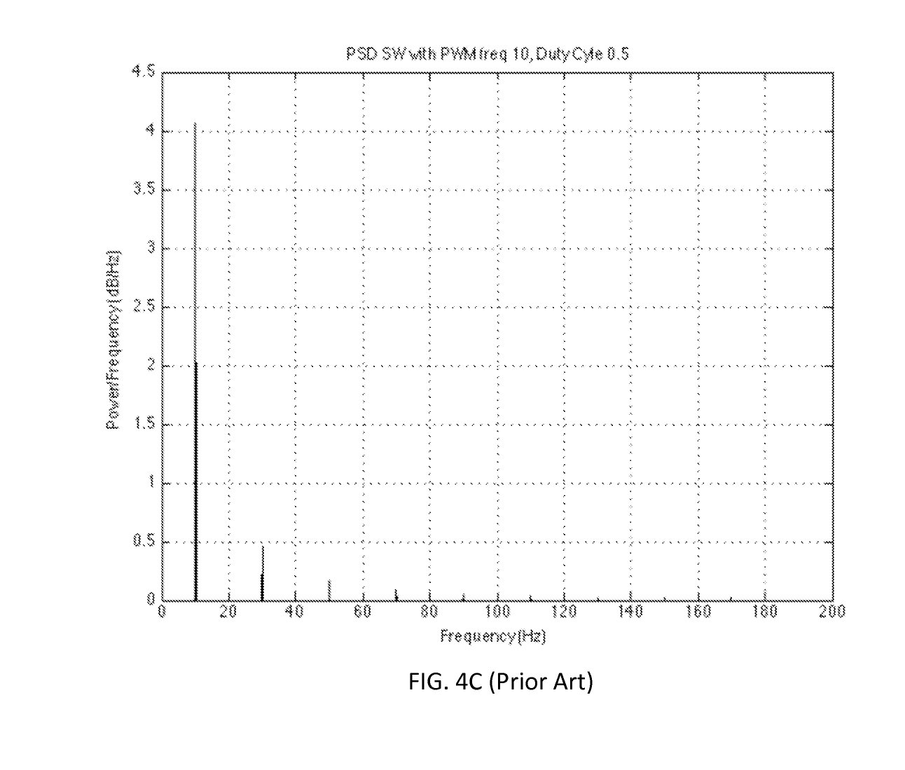

FIGS. 3A, 3B, and 3C illustrate various pulse waves with a frequency of 10 Hz and a duty cycle varying from 0.1 up to 0.5. Since the impedance of the system is fixed, the total amount of current (or alternatively voltage) reaching the nervous system decreases with decreasing the duty cycle. There is substantial evidence in the basic neuroscience literature that the nervous system encodes relevant features (such as motor plans, and pain perception) through a complex frequency code (Sachs et al., 2011; Miller et al., 2010; Elder and Sachs, 2004; Brittain and Brown, 2014; Tsubo et al., 2013; Schulz et al., 2011). There is also clinical evidence supporting different responses to different frequencies of stimulation (Sachs et al., 2014). In the clinical setting, the frequency of stimulation is programmed to optimize benefit to the patient. However, there are significant limitations to the frequency content of current stimulation systems using pulse waves with a fixed (programmable) frequency. FIGS. 4A, 4B, and 4C show the power spectral density of pulse wave with frequency 10 Hz and duty cycle varying from 0.1 to 0.5. This is a representation of frequency content of waveforms, and is analogous to the harmonic spectrum in music. The highest peak of the power spectrum occurs at the frequency of the waveform (the “fundamental frequency”). Additional frequency content occurs at integer multiples of the fundamental frequency. Lowering the duty cycle increases the amount of high frequency content in the power spectrum. This results in two fundamental limitations to existing systems: (1) the frequency content is limited to integer multiples of the fundamental frequency, and (2) in order to increase higher frequency content, the user must either decrease the duty cycle, which results in reducing the overall current applied to the nervous system, or increase the frequency (i.e., the fundamental frequency) of the stimulator in which they lose the potential benefit of the lower frequencies.

The present invention describes four inventive approaches used to address the shortcomings of the art, described in the following paragraphs as Solutions 1 through 4.

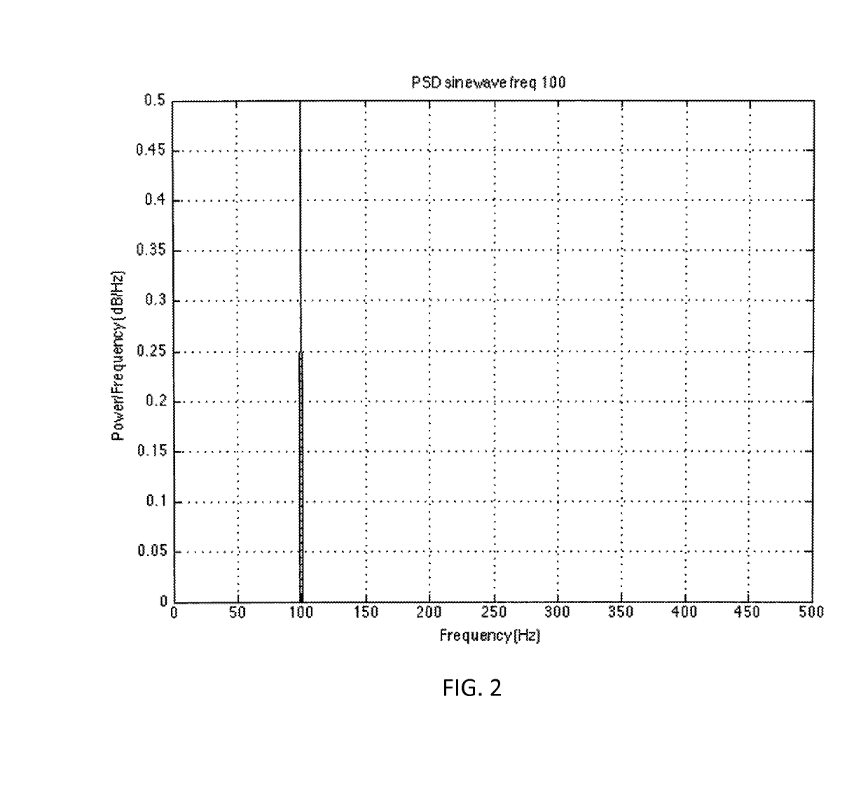

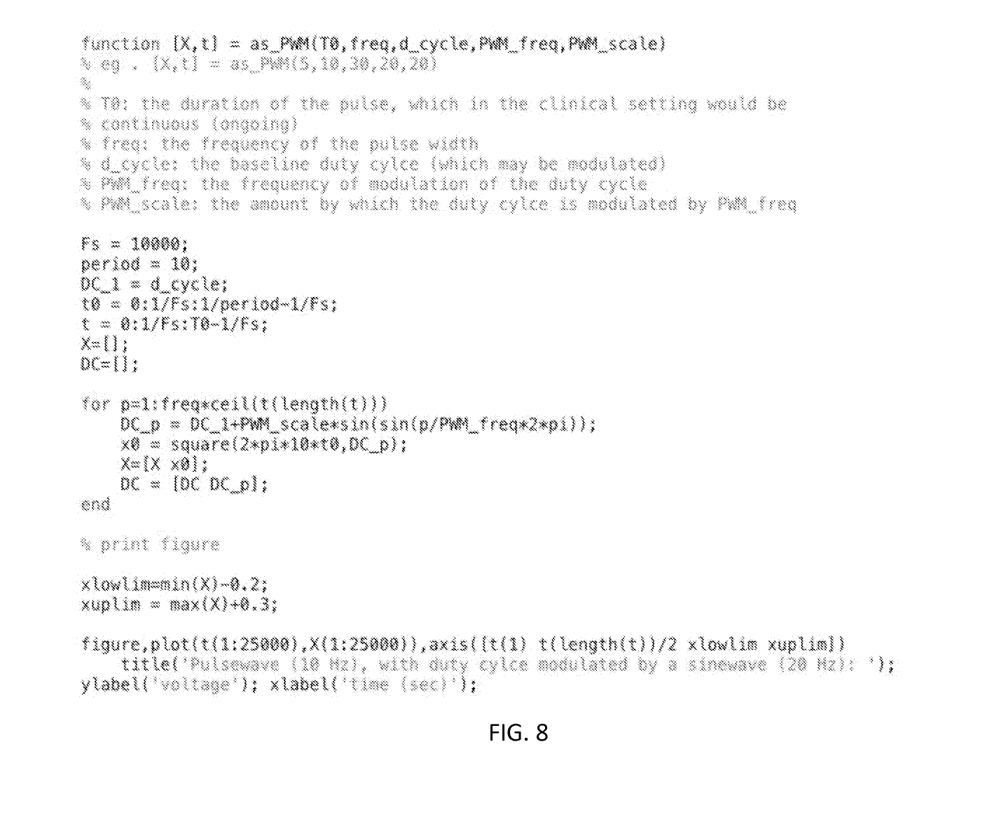

In Solution 1, a starting signal is produced, which, in certain embodiments, may be a pulse wave, a sine wave, a triangle wave, a sawtooth wave or a ramp (reverse sawtooth). An example sine wave is shown in FIG. 2. Solutions 2-4 offer methods of modulating these starting signals. An oscillator generates a signal continuously, and the parameters of the signal may be controlled. These signals and subsequent processing may be replicated digitally. For example, FIG. 8 provides Matlab™ code to generate these five fundamental waveforms.

Solution 2 refers to a technique that can be used to shape the frequency spectrum of the stimulation by using the addition of multiple signals with varying waveforms, amplitudes and frequencies. This can be expressed mathematically as:

$$f ( t ) = {a}_{0} + {\sum }_{k = 1}^{n} {a}_{k} {Ψ}_{k} ( {b}_{k} π t ) ,$$

where f(t) represents the voltage applied to the tissue, a₀, represents a direct current (dc) offset, n is the number of waveforms being added, ψₖ( ) represents each individual waveform, which may be a sinewave, squarewave, or other waveforms of solution 1, and may also incorporate pulse width and frequency modulation. Furthermore, aₖ represents the amplitude of each waveform and may be negative, and bₖ represents the frequency of each waveform. By controlling the parameters, using an external controller, the final stimulus, f(t), will have a frequency spectrum that can be tailored to optimize each patient's symptom and side effect profile. For example, increasing amplitude with increase the tissue volume activated of that waveform, and different neural networks tend to respond to different frequency ranges in different ways. In addition, different frequency ranges can change the tissue penetration profile.

In Solution 3, lowpass, bandpass and highpass filtering may be used to restrict certain frequencies and permit others in the final stimulation signal. For example, a low-pass filter prevents transmission of frequencies above a certain frequency. A band-pass filter permits frequencies within a certain range to pass through, while filtering the high and low signals out. With analog modulation, the cut-off is gradually increasing, rather than an abrupt cut-off, resulting in a gentler clipping. In addition, the filter cutoff(s) may be modulated by one of the waveforms described in Solution 1. Typically this would be done at a lower frequency.

In a preferred embodiment, a Butterworth filter may be used. The Butterworth filter rolls off more slowly around the cutoff frequency than the Chebyshev filter or the Elliptic filter, but without ripple, and has a flat frequency response in a passband. In alternative embodiments, Chebyshev Type I/Type II and elliptic filters can be used. The basic filter type is low pass, but highpass and bandpass can be achieved through various analog-digital transformations, including impulse invariance and bilinear transformation. Multiple stop/pass bands can be achieved by combining multiple filters with single pass band. This would allow the generated signal to have a smoother frequency spectrum, which better replicates the endogenous signals of the nervous system.

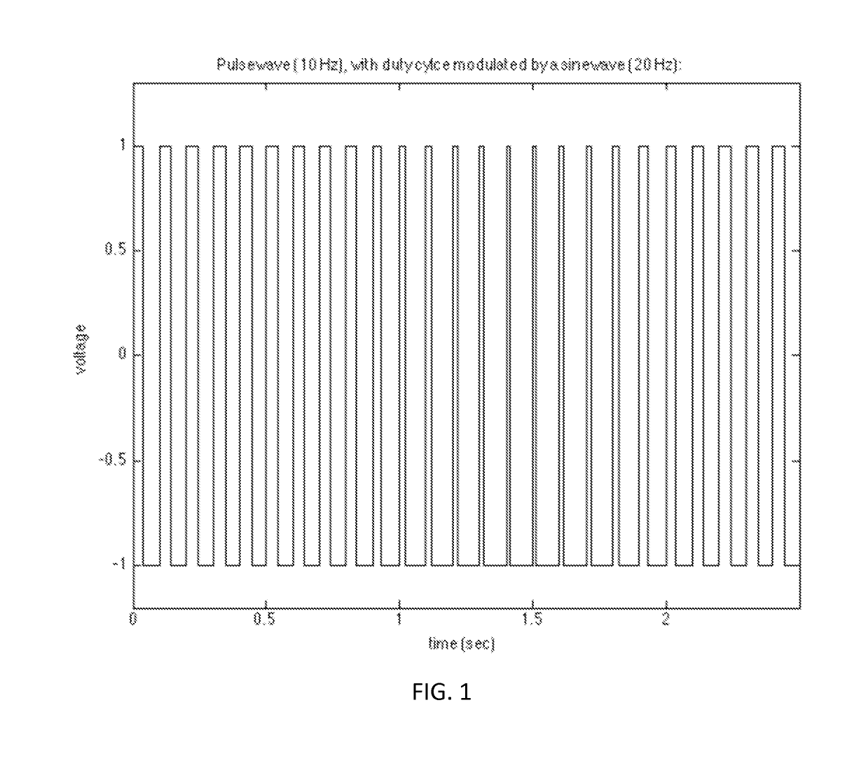

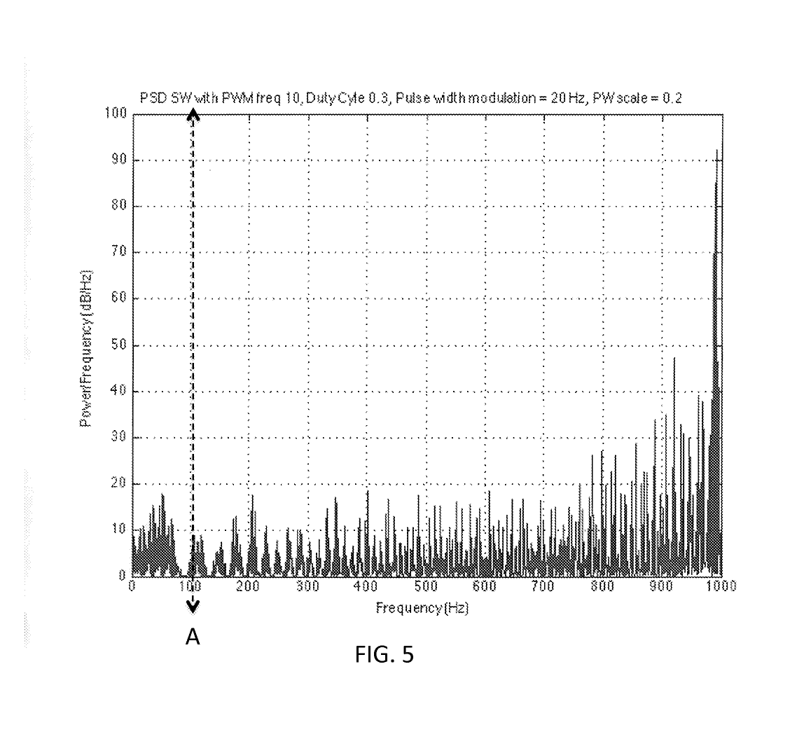

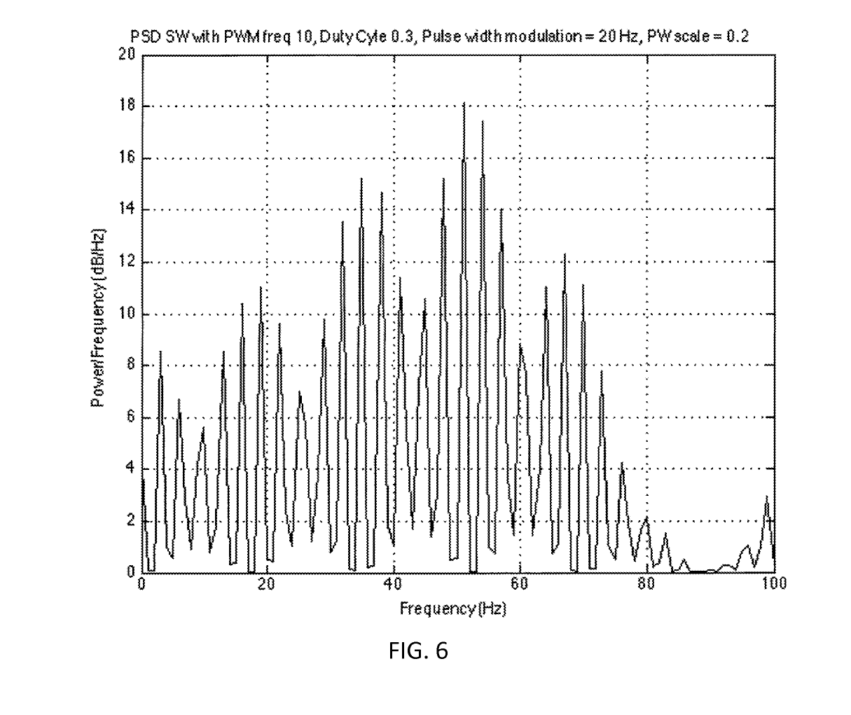

In Solution 4, the pulse width of a pulsewave may be modulated by one of the waveforms in Solution 1. An embodiment of this is shown in FIG. 1. Example Matlab™ code is shown in FIG. 8 for pulse-width modulation of a signal. This results in a significantly richer and broader frequency spectrum, wherein example waveforms are shown in FIGS. 5 and 6.

Solutions 1 to 4 provide treatment option for neuromodulation, in order to provide a rich frequency and amplitude range for treatment of the nervous tissue. Due to the irregular firing behaviors of neural networks naturally having highly irregular spectral densities with modulating frequency content, effective treatment must be capable of producing current that can reflect this frequency-domain complexity, which may be effected by Solutions 1 to 4. Once the signal is generated in accordance with Solutions 1, 2, 3 or 4, the signal is transmitted to the nervous tissue through electrodes that are placed on the tissue at issue.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A method of producing signals for neuromodulation, comprising the steps of:
a. producing a starting digital waveform; and
b. shaping the frequency spectrum of the signal, wherein the signal is configured to be used for neuromodulation treatment.

2. The method of claim 1, wherein the starting signal is selected from the group consisting of a pulse wave, a sine wave, a triangle wave, a sawtooth wave and a reverse sawtooth wave.

3. The method of claim 1, wherein the starting signals may be added, and the frequency spectrum of the signal is shaped according to the formula:
$$f ( t ) = {a}_{0} + {\sum }_{k = 1}^{n} {a}_{k} {Ψ}_{k} ( {b}_{k} π t ) ,$$

4. The method of claim 1, wherein the frequency spectrum of the signal is shaped by pulse width modulation.

5. The method of claim 1, wherein the frequency spectrum of the signal is shaped by frequency modulation.

6. The method of claim 1, further comprising the step of applying a filter to the signal.

7. The method of claim 6 wherein the filter is selected from the group consisting of lowpass filters, bandpass filters and highpass filters, and the parameters of the filters may be modulated.

8. The method of claim 6 wherein the filter is selected from the group consisting of Butterworth filters, Chebyshev Type I filters, Chebyshev Type II filters and elliptic filters, and the parameters of the filters may be modulated.

9. The method of claim 1, further comprising the step of applying pulse-width modulation to the signal.
